(19) United States
(12) Patent Application Publication
(10) Pub. No.: US 2012/0165732 A1
(43) Pub. Date: Jun. 28, 2012

(54) BALLOON CATHETER COMPRISING A ZERO-PROFILE TIP

(75) Inventor: Marc Müller, Weil am Rhein (DE)

(73) Assignee: Synthes USA, LLC, West Chester, PA (US)

(21) Appl. No.: 12/978,223

(22) Filed: Dec. 23, 2010

(51) Int. Cl. A61M 29/00

(52) U.S. Cl. 604/99.01

(57) ABSTRACT

Described herein is a balloon catheter with a zero-profile tip—that is, a balloon catheter having a distal tip that does not extend beyond the boundary of the cavity that will be created by the balloon when inflated—and further described herein are methods for the manufacturing of same. Several embodiment feature a method for inverting the distal end of an inflatable balloon structure, said inflatable balloon structure having a middle region, a first end region with a first opening, and a second end region with a second opening, said method comprising: (1) centrally inverting the second end region of the inflatable balloon structure and passing it through the first opening; (2) permanently fixing the inverted second end region to prevent un-inversion; and (3) returning the second end region back through the first opening.

Vertebral compression fractures represent a significant portion of all spinal injuries and can result from osteopororsis, metastatic diseases, or from trauma to the spine. Often vertebral compression fractures are treated using a minimally invasive posterior transpedicular or extrapedicular approach to perform vertebroplasty or kyphoplasty. Vertebroplasty is where a medical-grade bone cement (such as polymethylmethacrylate, a.k.a., PMMA) is injected percutaneously via a catheter into a fractured vertebra with the goal of relieving the pain stemming from the vertebral compression fractures. Kyphoplasty is a variation of a vertebroplasty that further attempts to restore the height and angle of kyphosis of a fractured vertebra using a balloon-like structure at the distal end of a catheter that is inflated in the vertebral body to create a cavity to contain the delivery of bone cement or other spacing material. Procedurally, kyphoplasty involves making small incisions and placing the balloon catheter into the vertebral space such that the balloon can be expanded to create a cavity inside the bone where the bone cement will be added after deflating and removing the balloon catheter.

Generally, the distal end of a kyphoplasty balloon catheter comprises an insertion tip that extends some distance beyond the distal end of the uninflated balloon and, as such, this tip must be introduced into the vertebral body a distance beyond the boundary of the cavity that will be created by the balloon when inflated in order to ensure that the balloon is properly positioned within the vertebral body. Consequently, there is a risk that the tip can extend too far and damage the anterior wall of the vertebral body, especially since it is desirable to place the balloon as near as possible to the anterior wall to achieve an optimum filling and maximum restoration of the height of the vertebral body. Moreover, the tip itself creates a dead space near the distal portion of the balloon whereby the amount of dead space is related to how far the tip extends beyond the distal end of the balloon.

To overcome these shortcomings, a balloon catheter with a zero-profile tip—that is, a balloon catheter having a distal tip that does not extend beyond the boundary of the cavity that will be created by the balloon when inflated—can be used to create a cavity in a vertebral body. However, existing balloon catheters having zero-profile tips suffer from several design shortcomings in their deployable configuration or in the complexity of their manufacture.

The present disclosure relates generally to orthopedics. More specifically, the present disclosure relates to balloon catheters. Described herein is a balloon catheter with a zero-profile tip—that is, a balloon catheter having a distal tip that does not extend beyond the boundary of the cavity that will be created by the balloon when inflated—and methods for the manufacturing of same.

Disclosed herein are embodiments of a zero-profile tip balloon catheter substantially comprising: (a) an inflatable balloon structure comprising a middle region, a proximal end region with a proximal opening, and a distal end region with a distal opening, wherein the distal end region is proximally invertible and passable through said proximal opening, and wherein the middle region and distal end region are sufficiently flexible to permit the distal end region to be inverted and passed through the proximal opening; and (b) a catheter comprising a distal end and circumferentially connectively coupled to said inflatable balloon structure at both the proximal end region and the distal end region such that the distal end region of the inflatable balloon structure distally extends beyond the distal end of the catheter, wherein the distal end region is inverted where connectively coupled to said catheter.

Further disclosed herein are methods of manufacturing a zero-profile tip balloon catheter, said zero-profile balloon tip catheter comprising a catheter and an inflatable balloon structure having a middle region, a proximal end region with a proximal opening, and a distal end region with a distal opening, said methods substantially comprising: (i) centrally inverting the distal end region of the inflatable balloon structure and passing it through the proximal opening; (ii) with the distal end region still inverted, introducing a catheter into the distal opening of the distal end region; (iii) with the distal end region still inverted, connectively coupling the catheter and the distal end region at a first coupling location; (iv) returning the distal end region, now connectively coupled to the catheter and still inverted where connectively coupled to the catheter, back through the proximal opening; and (v) connectively coupling the proximal end region to the catheter at a second coupling location on the catheter proximal to the first coupling location.

Further disclosed is a method for inverting the distal end of an inflatable balloon structure, said inflatable balloon structure having a middle region, a first end region with a first opening, and a second end region with a second opening, said method comprising: (1) centrally inverting the second end region of the inflatable balloon structure and passing it through the first opening; (2) permanently fixing the inverted second end region to prevent un-inversion; and (3) returning the second end region back through the first opening.

To facilitate an understanding of and for the purpose of illustrating the present disclosure, exemplary features and implementations are disclosed in the accompanying drawings, it being understood, however, that the present disclosure is not limited to the precise arrangements and instrumentalities shown, and wherein similar reference characters denote similar elements throughout the several views, and wherein:

FIG. 1 is a cross-sectional view of a first balloon catheter with a zero-profile tip;

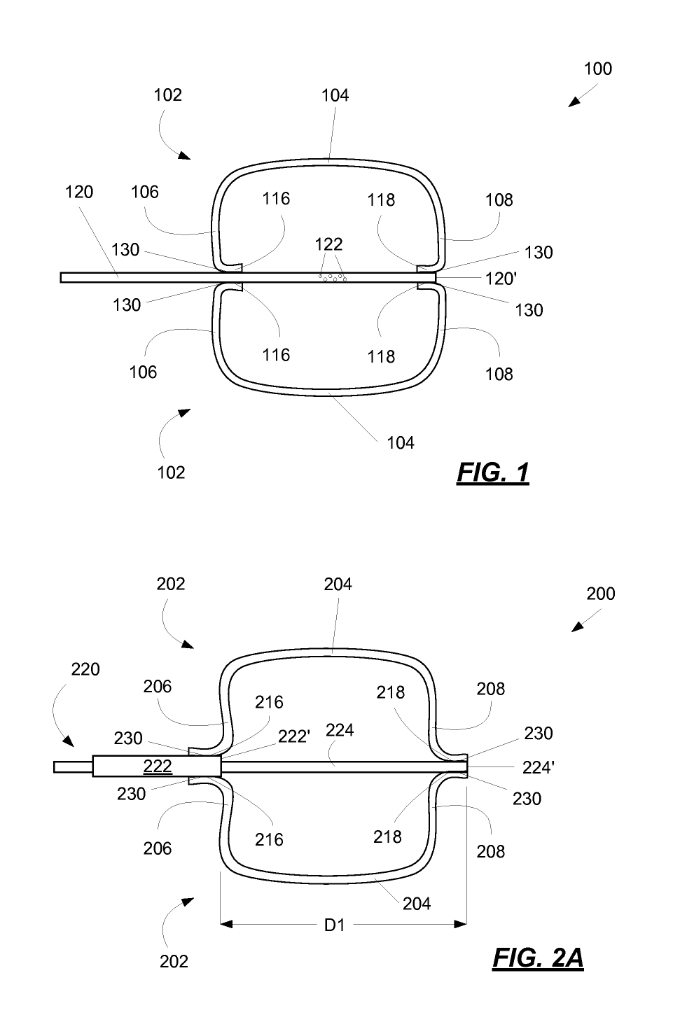

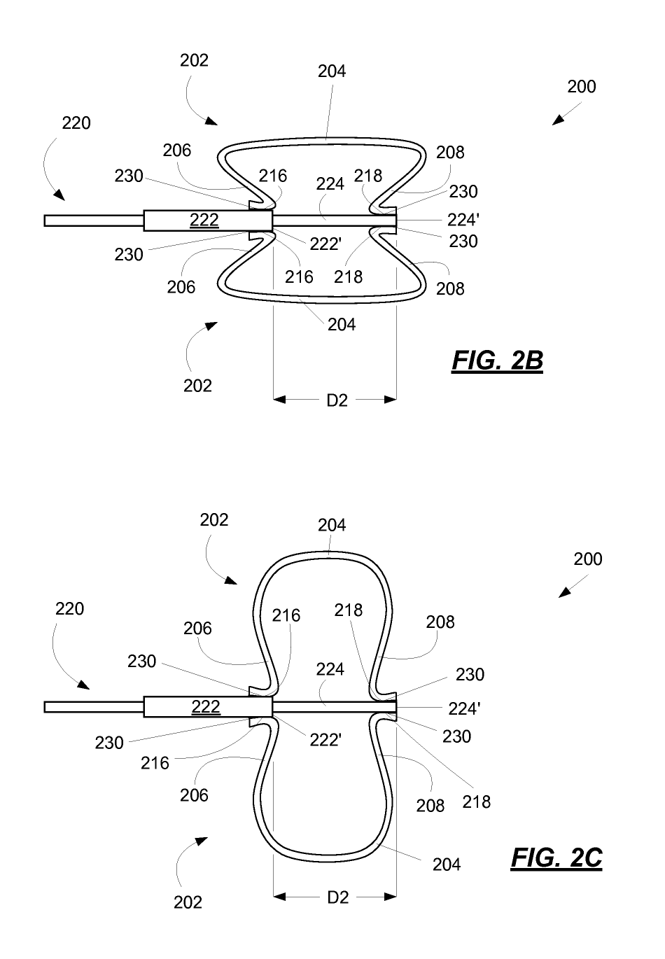

FIG. 2A is a cross-sectional view of a second balloon catheter with a zero-profile tip with the distal end of the balloon structure in a non-inverted state for manufacture;

FIG. 2B is a cross-sectional view of the second balloon catheter with a zero-profile tip of FIG. 2A with the distal end of the balloon structure in an ideally inverted state during utilization;

FIG. 2C is a cross-sectional view of the second balloon catheter with a zero-profile tip of FIG. 2A that has failed to achieve an ideally inverted state;

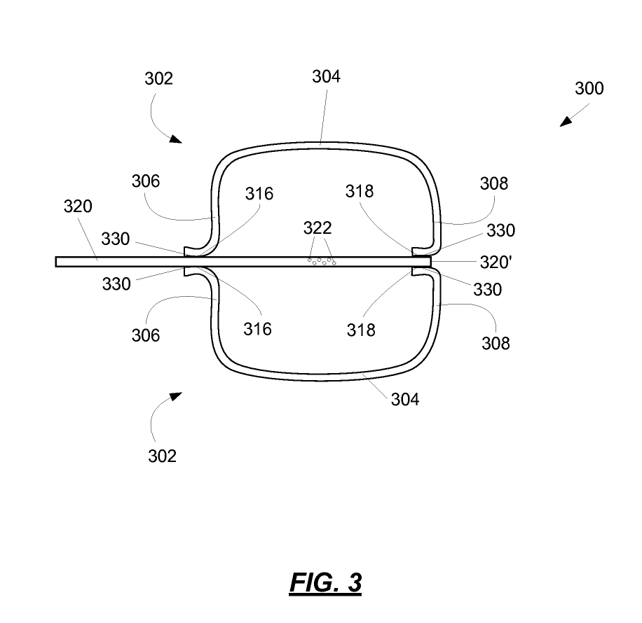

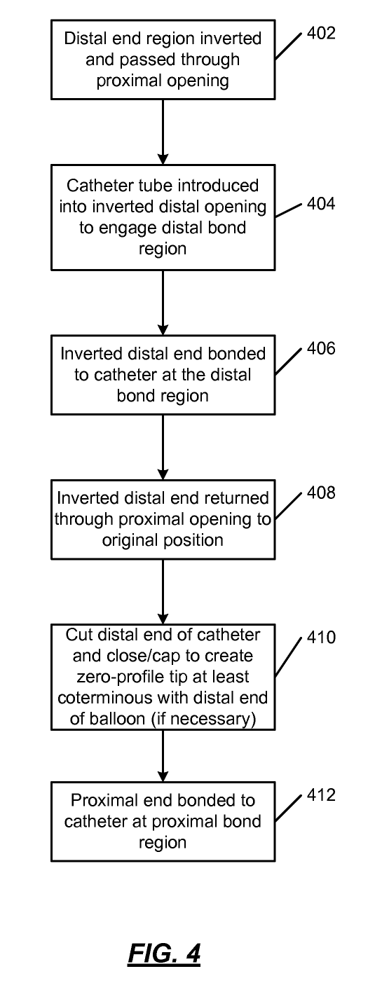

FIG. 3 is a cross-sectional view of a third balloon catheter with a zero-profile tip;

FIG. 4 is an operational flow diagram of exemplary processes to form the third balloon catheter with a zero-profile tip of FIG. 3.

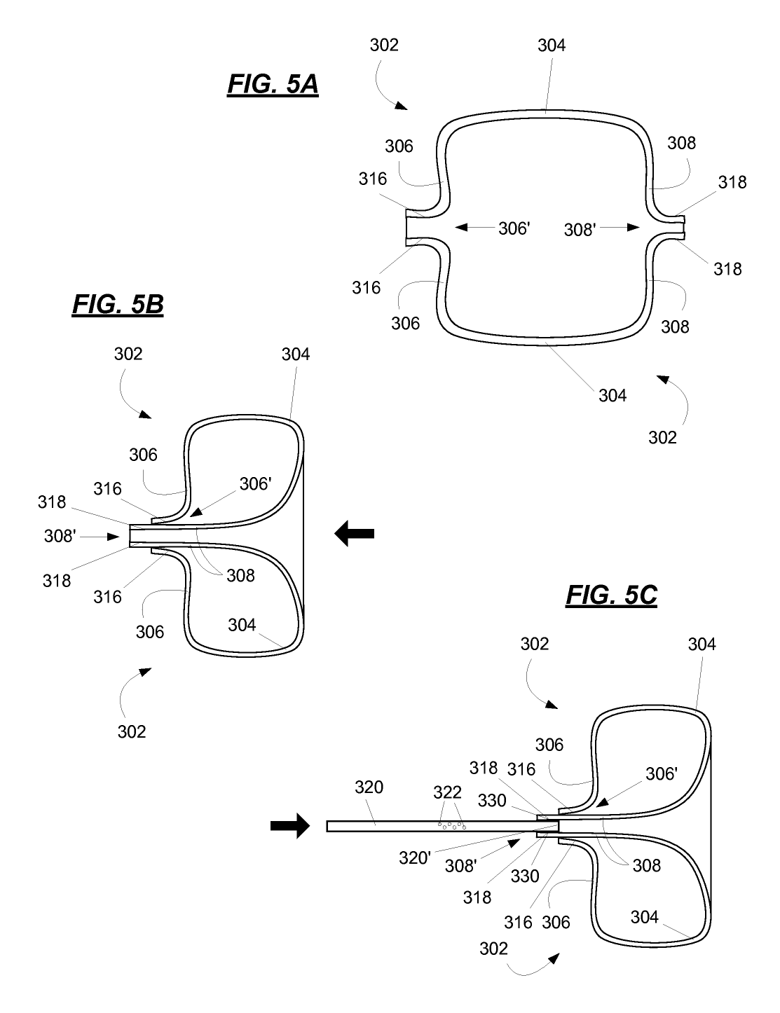

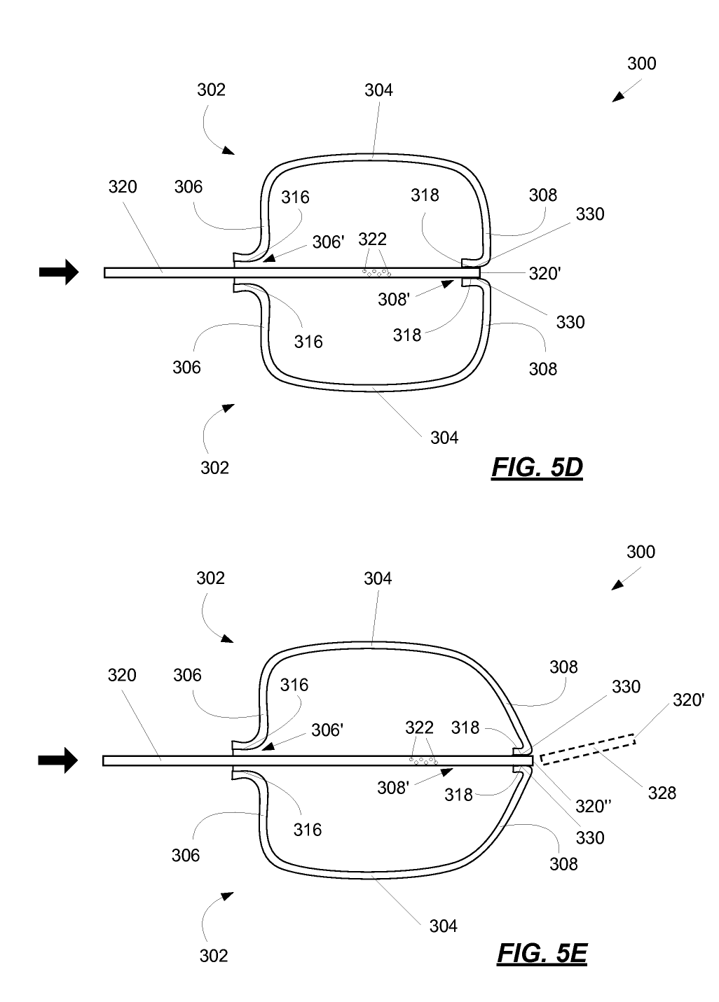

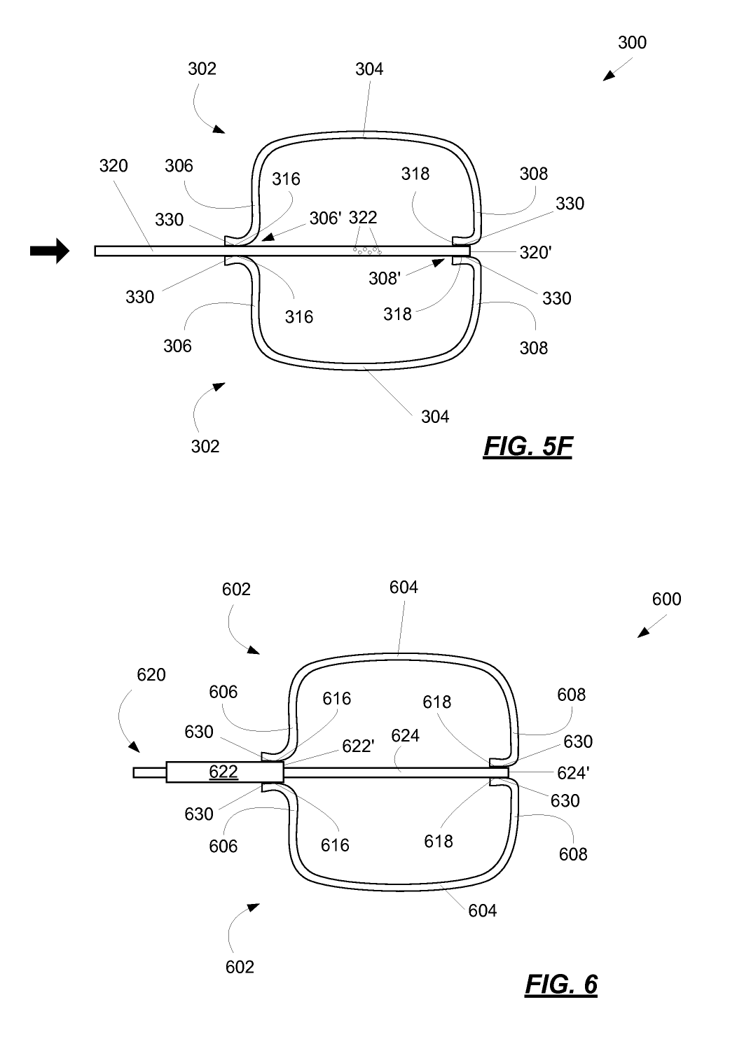

FIGS. 5A-5F are cross-sectional views of the third balloon catheter with a zero-profile tip of FIG. 3 during manufacture in accordance with the steps described in FIG. 4.

FIG. 6 is a cross-sectional view of fourth balloon catheter with a zero-profile tip featuring a double-lumen catheter; and

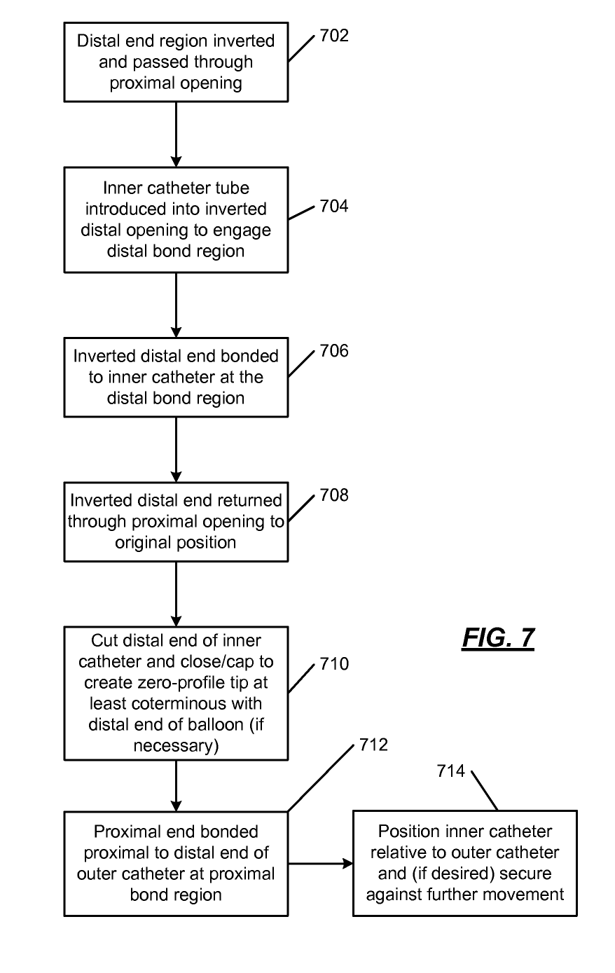

FIG. 7 is an operational flow diagram of exemplary processes to form the fourth balloon catheter with a zero-profile tip featuring a double-lumen catheter of FIG. 6.

A balloon catheter with a zero-profile tip—that is, a balloon catheter having a distal tip that does not extend beyond the boundary of the cavity that will be created by the balloon when inflated—can be used to create a cavity in a vertebral body. FIG. 1 is a cross-sectional view of a first balloon catheter with a zero-profile tip 100 comprising an inflatable balloon structure 102 that includes a middle region 104, a fully inverted proximal end region 106 comprising a proximal bond region 116, and a fully inverted distal end region 108 comprising a distal bond region 118. Both the proximal end 106 and the distal end 108 of the balloon 102 are mechanically tucked or folded inward and placed into contact with a catheter tube 120 such that at least the distal end 120′ of the catheter tube 120 does not extend beyond the distal end 108 of the balloon 102. The distal end 120′ of the catheter 120 may be closed off or capped, while the catheter 120 possesses inflation vents 122 between the proximal bond region 116 and the distal bond region 118 through which an inflatable medium (not shown) can flow to inflate and/or deflate the balloon 102. The structure 100 comprises, when substantially collapsed, a tube-like structure with a total thickness at the distal end equal to the width of the catheter tube 120 plus four times (4×) the thickness of the balloon 102 material, said material being doubled-over at the contact points with the catheter tube 120. p In manufacturing this first balloon catheter 100, the catheter tube 120 might first be coated with a welding material 130 at locations on the catheter tube 120 corresponding to the anticipated placement of the proximal bond region 116 and the distal bond region 118 of the balloon 102. Then, after the folded-inward proximal bond region 116 and distal bond region 118 of the balloon 102 are brought into abutment against the catheter tube 120, welding energy (such as microwave or laser energy) can be transmitted from an external source (not shown) through the middle region 104 and absorbed by the welding material 130 to form a weld—with the welding material 130 having substantially zero thickness of its own—between (a) the folded-inward proximal bond region 116 and distal bond region 118 of the balloon 102 and (b) the catheter tube 120. Maximal distal placement of the folded-inward distal bond region 118 may achieve an abrupt termination of the distal end 108 of the balloon 102 adjacent the distal end 120′ of the catheter tube 120, such that the distal end region 108 and the distal end 120′ of the catheter tube 120 are coterminous.

A challenge of this approach, of course, is that the welding material 130 used in the manufacture of the device must be of a type that can absorb a specific welding energy that does not otherwise damage the balloon 102 or the catheter 120 in any way. In other words, since the welding energy might be transmitted from an external source through the balloon 102—such as the middle region 104 of the balloon 102—this welding energy must be of a type that will not damage the material of the balloon 102 through which it is passed. This inherently limits the type of material from which the balloon 102 can be made, as well as limits the type of welding material 130 that can be utilized in manufacturing the balloon catheter 100.

FIG. 2A is a cross-sectional view of a second balloon catheter with a zero-profile tip 200 with the distal end 208 of the balloon structure 202 in a non-inverted state for manufacture. The balloon catheter 200 comprises an inflatable balloon structure 202 that includes a middle region 204, a non-inverted proximal end region 206 comprising a proximal bond region 216, and a non-inverted distal end region 208 comprising a distal bond region 218. Both the proximal end 206 and the distal end 208 of the balloon 202 are placed into contact with a double lumen catheter 220 comprising an outer catheter tube 222 and an inner catheter tube 224 such that the inner catheter tube 224 slides within the outer catheter tube 222 and where the distal end 224′ of the inner catheter tube 224 extends beyond the distal end 222′ of the outer catheter tube 222. The proximal bond region 216 of the proximal end 206 of the balloon 202 is placed into contact with the outer catheter tube 222 near its distal end 222′, while the distal bond region 218 of the distal end 208 of the balloon 202 is placed into contact with the inner catheter tube 224 near its distal end 224′. The outer catheter tube 222 can be used to inflate and deflate the balloon 202, while the inner catheter tube 224 can be closed or capped, or left open for another catheter-specific purpose.

During the manufacturing process, the distal end 224′ of the inner catheter tube 224 is moved a first distance D1 beyond the distal end 222′ of the outer catheter tube 222. In this configuration, the proximal end 206 and the distal end 208 of the balloon structure 202 are bonded, without folding inward, about the outer catheter tube 222 and the inner catheter tube 224 at the proximal bond region 216 and the distal bond region 218 respectively, using any form of suitable adhesive, melt-bonding process, or other bonding method, such that the material used to form the bond 230 has substantially zero thickness of its own.

FIG. 2B is a cross-sectional view of the second balloon catheter with a zero-profile tip of FIG. 2A with the distal end of the balloon structure in an ideally inverted state during utilization. With regard to FIG. 2B, and once the bonds 230 at the bonded regions 216 and 218 are formed in the configuration shown in FIG. 2A, the inner catheter tube is then moved to a distance D2 (shorter than D1) such that the shorting of the distance between the distal end 224′ of the inner catheter tube 224 and the distal end 222′ of the outer catheter tube 222 double-inverts the ends 206 and 208 of the balloon 202 to create double jointed overlaps as shown in FIG. 2B such that the double jointed overlaps of the ends 206 and 208 overlie the bonded regions 216 and 218 and the distal end 224′ of the inner catheter tube 224 does not extend beyond the distal end 208 of the balloon 102.

The catheter 200 comprises, when substantially collapsed, a tube-like structure with a total thickness at the distal end equal to the width of the inner catheter tube 224 plus six times (6×) the thickness of the balloon 202 material, said material being twice inverted to form two doubled-overs (i.e., a triple-over) proximate to the contact points with the inner catheter tube 224. This additional thickness and resulting larger circumferential profile of the structure 200 require a larger incision in the patient to emplace the device 200, which may be relatively undesirable versus a smaller incision.

It should also be noted that for this catheter 200 the balloon 202 must be specially shaped and pre-formed, possibly using specialized materials or thicker portions of the same material as the rest of the balloon 202, such that the middle region 204 is substantially rigid in order for the shorting of the distance between the distal end 224′ of the inner catheter tube 224 and the distal end 222′ of the outer catheter tube 222 to result in double-inverting the ends 206 and 208 of the balloon 202 to create double jointed overlaps of the ends 206 and 208 that overlie the bonded regions 216 and 218 such that the distal end 224′ of the inner catheter tube 224 does not extend beyond the distal end 208 of the balloon 102. If the middle region is of a greater thickness, this additional thickness and resulting larger circumferential profile of the structure 200 may also require an even larger incision in the patient to emplace the device 200, which may be even more undesirable for certain patients. Likewise, if the middle region 204 of the balloon is made from special materials, these materials may be more costly or difficult to work with or complex in their manufacture.

Significantly, without a substantially rigid middle region 204 the balloon may not expand as desired but might instead distend more equally along its entire surface such as show in FIG. 2C whereby the shorting of the distance between the distal end 224′ of the inner catheter tube 224 and the distal end 222′ of the outer catheter tube 222 would not result in double-inverting the ends 206 and 208 of the balloon 202 and, consequently, the distal end 224′ of the inner catheter tube 224 would continue to extend beyond the distal end 208 of the balloon 102. FIG. 2C is a cross-sectional view of the balloon catheter of FIGS. 2A and 2B that has failed to achieve an inverted state and therefore lacks a zero-profile tip.

A third balloon catheter with a zero-profile tip is herein disclosed and illustrated in FIG. 3. The balloon catheter 300 comprises an inflatable balloon structure 302 that includes a middle region 304, a non-inverted proximal end region 306 comprising a proximal bond region 316, and a fully inverted distal end region 308 comprising a distal bond region 318. The proximal end 306 and the distal end 308 of the balloon are in contact with the catheter tube 320 such that at least the distal end 320′ of the catheter tube 320 does not extend beyond the distal end 308 of the balloon 302. For a single-lumen catheter (as shown herein FIG. 3), the distal end 320′ of the catheter 320 may be closed off or capped, while the catheter 320 may possess inflation vents 322 located between the proximal bond region 116 and the distal bond region 118 through which an inflatable medium (not shown) can flow to inflate and/or deflate the balloon 102. The structure 300 comprises, when substantially collapsed, a tube-like structure with a total thickness at the distal end equal to the width of the catheter tube 320 plus four times (4×) the thickness of the balloon 302 material, said material being doubled-over at the distal bond region 318.

FIG. 4 is an operational flow diagram of exemplary processes to form the embodiment of the third balloon catheter with a zero-profile tip of FIG. 3. FIGS. 5A-5F are cross-sectional views of the third balloon catheter with a zero-profile tip of FIG. 3 during manufacture in accordance with the steps described in FIG. 4. With reference to FIGS. 3, 4, and 5A-5F, the manufacture and assembly of the third balloon catheter 300 first comprises the inflatable balloon structure 302 that includes a middle region 304, a proximal end region 306 comprising a proximal opening 306′ and having a proximal bond region 316 on the interior surface of the proximal end region 306, and a distal end region 308 with a distal opening 308′ comprising a distal bond region 318 on the exterior surface of the distal end region 308, as shown in FIG. 5A. At step 402 and as shown in FIG. 5B, the distal end region 308 is centrally inverted and passed through the proximal opening 306′ such that the distal end region 308 extends beyond the proximal end region 306 a distance at least roughly equivalent to the length of the distal bond region 318. At step 404 and as shown in FIG. 5C, distal end 320′ of the catheter tube 320 is introduced into the inverted distal opening 308′ at least far enough to engage the distal bond region 318. For a single-lumen catheter (as shown) where the distal end 320′ of the catheter 320 is closed off or capped, and where the catheter 320 possesses inflation vents 322, the catheter tube 320 is introduced into the inverted distal opening 308′ such that said inflation vents 322 will ultimately be located central and internal to the balloon 302 between the proximal bond region 316 and the distal bond region 318 at the end of the assembly.

At step 406, the inverted distal end 308 of the balloon structure 302 is bonded to the catheter tube 320 at the distal bond region 318 using any form of suitable adhesive, melt-bonding process, or other bonding method, preferably such that the material used to form the bond 330 has substantially zero thickness of its own. Once the bond 330 is formed, at step 408 and as shown in FIG. 5D, the catheter tube 320 and balloon 302 are moved laterally with relation to each other in order to pass the inverted distal end 308 of the balloon 302 back through the proximal opening 306′ and return the distal end 308 toward its original position, now in a permanently inverted configuration where the distal bond region 318 lies within the inflatable balloon structure 302. In an alternative embodiment where the distal end 320′ of the catheter 320 extends beyond the distal bond region 318 of the distal end 308 of the balloon 302, then (at step 410 and as shown in FIG. 5E) the catheter 320 is cut to remove the excess catheter length 328 and closed to create new a distal end 320″ coterminous with the distal bond region 318. This can be achieved by temporarily extending the catheter 320 through the proximal opening 306′ of the proximal end 306 of the balloon 302 toward the distal end 308 of the balloon 302, thereby extending the distal bond region 318 to a position coterminous to the distal end 308 of the balloon 302 and accessible to a cutting device (not shown). Once the catheter is cut and closed (and excess 328 is removed) to create new distal end 320″, the catheter can then be retracted such that the distal bonded region 318 is once again favorably located internal to the balloon structure 302 thereby forming the desired zero-profile tip. At step 412 and as shown in FIG. 3F, the proximal end 306 of the balloon 302 is bonded to the catheter tube 320 at the proximal bond region 316 using any form of suitable adhesive, melt-bonding process, or other bonding method, preferably such that the material used to form the bond 332 has substantially zero thickness of its own. Moreover, for certain embodiments, the middle region, proximal end region, and distal end region of the inflatable balloon structure can comprise the same material composition and do not require, for example, specially materials for a stiff structure of said middle region.

FIG. 6 is a cross-sectional view of a fourth balloon catheter with a zero-profile tip. Similar to the embodiment illustrated in FIG. 3, the balloon catheter 600 comprises an inflatable balloon structure 602 that includes a middle region 604, a non-inverted proximal end region 606 comprising a proximal bond region 616, and a fully inverted distal end region 608 comprising a distal bond region 618. For this embodiment, however, the proximal end 606 and the distal end 608 of the balloon are in contact with a double-lumen catheter 620 comprising an outer catheter tube 622 and an inner catheter tube 624 such that the inner catheter tube 624 slides within the outer catheter tube 622 and where the distal end 624′ of the inner catheter tube 624 extends beyond the distal end 622′ of the outer catheter tube 622. The proximal bond region 616 of the proximal end 606 of the balloon 602 is in contact with the outer catheter tube 622 near its distal end 622′, while the distal bond region 618 of the distal end 608 of the balloon 602 is in contact with the inner catheter tube 624 near its distal end 624′. The outer catheter tube 622 can be used to inflate and deflate the balloon 602, while the distal end 624′ of the inner catheter tube 624 can be closed or capped. In alternative embodiments, the distal end 624′ of the inner catheter tube 624 could be left open for another catheter-specific purpose. The double-lumen catheter 620 may be a double-lumen catheter, such as that disclosed in U.S. patent application Ser. No. 12/904,975, which is incorporated herein by reference in its entirety.

FIG. 7 is an operational flow diagram of exemplary processes to form the fourth balloon catheter with a zero-profile tip of FIG. 6. With reference to FIGS. 6 and 7, the manufacture and assembly of the fourth balloon catheter 600 first comprises the inflatable balloon structure 602 (similar to the balloon structure illustrated in FIG. 5A). At step 702, the distal end region 608 of the balloon 602 is centrally inverted and passed through the proximal opening 606′ such that the distal end region 608 extends beyond the proximal end region 606 a distance at least substantially equivalent to the length of the distal bond region 618 (similar to the configuration illustrated in FIG. 5B). At step 704, the distal end 624′ of the inner catheter tube 624 of the double-lumen catheter 620 is introduced into the inverted distal opening 608′ at least far enough to engage the distal bond region 618 (similar to the configuration illustrated in FIG. 5C).

At step 706, the inverted distal end 608 of the balloon structure 602 is bonded to the inner catheter tube 624 at the distal bond region 618 using any form of suitable adhesive, melt-bonding process, or other bonding method, preferably such that the material used to form the bond 630 has substantially zero thickness of its own. Once the bond is formed, at step 708 (and similar to the configuration shown in FIG. 5D), the inner catheter tube 624 and balloon 602 are moved laterally with relation to each other in order to pass the inverted distal end 608 of the balloon 602 back through the proximal opening 606′ and return the distal end 608 toward its original position, now in a permanently inverted configuration where the distal bond region 618 lies within the inflatable balloon structure 602. In an alternative embodiment where the distal end 624′ of the inner catheter tube 624 extends beyond the distal bond region 618 of the distal end 608 of the balloon 602, then at step 710 (and similar to the configuration shown in FIG. 5E) the inner catheter tube 624 may be cut and closed (and excess removed) to create new a distal end 624″ (not shown, but corresponding to element 320″ in FIG. 5E) coterminous with the distal bond region 618. This can be achieved by temporarily extending the inner catheter tube 624 through the proximal opening 606′ of the proximal end 606 of the balloon 602 toward the distal end 608 of the balloon 602, thereby extending the distal bond region 618 to a position at least coterminous to the distal end 608 of the balloon 602 and/or accessible to a cutting device (not shown). Once the inner catheter tube 624 is cut and closed to create new distal end 620″ (not shown), the inner catheter tube 624 can then be retracted such that the distal bonded region 618 is once again favorably located internal to the balloon structure 602 thereby forming the desired zero-profile tip. At step 712 (and similar to the configuration shown in FIG. 3F), the proximal end 306 of the balloon 302 is bonded to in proximity to the distal end 622′ of the outer catheter tube 622 at the proximal bond region 616 using any form of suitable adhesive, melt-bonding process, or other bonding method, preferably such that the material used to form the bond 630 has substantially zero thickness of its own. In an alternative process, this step may be performed immediately after step 708 and before step 710. For certain embodiments, at step 714 the inner catheter tube 624 is then extend or retracted to a desired fixed position with regard to the outer catheter tube 622 and the balloon structure 602, and at step 716 the relative positions of the inner catheter tube 624 and outer catheter tube 622 are secured against further movement, e.g. by adhesive, etc., to complete the assembly of the structure 600.

In certain alternative embodiments of the balloon catheters with a zero-profile tip disclosed herein, such embodiments may include a balloon wherein the proximal opening is larger than the distal opening in order to facilitate an easier pass-through of the inverted distal end of the balloon through the proximal end of the balloon as described herein.

Moreover, the foregoing techniques can be applied to several other embodiments of devices for a variety of purposes featuring an inflatable balloon structure—whereby the method for inverting the distal end of an inflatable balloon structure, said inflatable balloon structure having a middle region, a first end region with a first opening, and a second end region with a second opening, said method comprising: (1) centrally inverting the second end region of the inflatable balloon structure and passing it through the first opening; (2) permanently fixing the inverted second end region to prevent un-inversion; and (3) returning the second end region back through the first opening—is anticipated by this disclosure.

Similarly, several aspects of the embodiments discussed herein are also possible in devices lacking a catheter and may instead comprise separate two components, one each at the distal end and the proximal end. For example, a zero-profile balloon device may simply comprise an inflatable balloon structure coupled to a proximal sealing component at the proximal end region and a distal sealing component at an inverted distal end region such that the distal end region of the inflatable balloon structure distally extends beyond the distal end of the distal sealing component. For such embodiments, the distal sealing component might comprise a plug or a tubular obstruction to seal off the inverted distal end, or a cap of some kind covering the inverted distal end. Similarly, the proximal sealing component might comprise a band, a collar, or a mechanical pinch of some kind to effectively seals off the proximal end. Such embodiments may also include a proximal sealing component having an inflation coupling for use in inflating or deflating the inflatable balloon structure, or even one or more inflation vent could be used. These embodiments might also have some kind of minimum spacing device (e.g., a buffer or bumper) internal to the inflatable balloon structure that maintains apart the distal sealing component and the proximal sealing component at some minimal distance (corresponding to the length of the spacing device). Conversely these embodiments might also have a maximum spacing device (e.g., a wire connected to each sealing component) internal to the inflatable balloon structure that maintains together the distal sealing component and the proximal sealing component at some maximum distance. Many other alternative embodiments and functional equivalents are likewise anticipated by this disclosure.

The subject matter described above is provided by way of illustration only and should not be construed as limiting. Various modifications and changes may be made to the subject matter described herein without following the example embodiments and applications illustrated and described, and without departing from the true spirit and scope of the present invention, which is set forth in the following claims.

1. A method of manufacturing a zero-profile tip balloon catheter, said zero-profile balloon tip catheter comprising a catheter and an inflatable balloon structure having a middle region, a proximal end region with a proximal opening, and a distal end region with a distal opening, said method comprising:
centrally inverting the distal end region of the inflatable balloon structure and passing it through the proximal opening;
with the distal end region still inverted, introducing a catheter into the distal opening of the distal end region;
with the distal end region still inverted, connectively coupling the catheter and the distal end region at a first coupling location;
returning the distal end region, now connectively coupled to the catheter and still inverted where connectively coupled to the catheter, back through the proximal opening; and
connectively coupling the proximal end region to the catheter at a second coupling location on the catheter proximal to the first coupling location.

2. The method of claim 1, wherein the catheter is a single-lumen catheter.

3. The method of claim 2, wherein the single-lumen catheter comprises a distal end, said method further comprising closing the distal end.

4. The method of claim 3, wherein said catheter comprises a plurality of inflation vents for inflating or deflating the inflatable balloon structure, wherein the first coupling location is relatively distal to the plurality of inflation vents, and wherein the second coupling location is relatively proximal on the plurality of inflation vents.

5. The method of claim 1, wherein the catheter is a double-lumen catheter comprising an outer catheter tube and an inner catheter tube, wherein the fully-inverted distal end region of the inflatable balloon structure is connectively coupled to the inner catheter tube, and wherein the proximal end region of the inflatable balloon structure is connectively coupled to the outer catheter tube.

6. The method of claim 5, further comprising closing the catheter tube at the distal end.

7. The method of claim 5, wherein the inner catheter tube is fixed to prevent movement within the outer catheter tube.

8. The method of claim 5, wherein the inner catheter tube is movable within the outer catheter tube to shorten or lengthen the distance between the proximal end region and the distal end region of the inflatable balloon structure.

9. The method of claim 1, wherein the proximal opening is larger than the distal opening.

10. A zero-profile tip balloon catheter comprising:
an inflatable balloon structure comprising a middle region, a proximal end region with a proximal opening, and a distal end region with a distal opening, wherein the distal end region is proximally invertible and passable through said proximal opening, and wherein the middle region and distal end region are sufficiently flexible to permit the distal end region to be inverted and passed through the proximal opening; and
a catheter comprising a distal end, said catheter connectively coupled to said inflatable balloon structure at both the proximal end region and the distal end region such that the distal end region of the inflatable balloon structure distally extends beyond the distal end of the catheter, wherein the distal end region is inverted where connectively coupled to said catheter.

11. The zero-profile tip balloon catheter of claim 10, wherein the catheter is a single-lumen catheter.

12. The zero-profile tip balloon catheter of claim 11, wherein the single-lumen catheter comprises a distal end, and wherein said single-lumen catheter is closed at the distal end.

13. The zero-profile tip balloon catheter of claim 12, wherein said catheter further comprises a plurality of inflation vents for inflating or deflating the inflatable balloon structure.

14. The zero-profile tip balloon catheter of claim 10, wherein the catheter is a double-lumen catheter comprising an outer catheter tube and an inner catheter tube, wherein the proximal end region of the inflatable balloon structure is connectively coupled to the outer catheter tube, and wherein the fully-inverted distal end region of the inflatable balloon structure is connectively coupled to the inner catheter tube.

15. The zero-profile tip balloon catheter of claim 14, wherein the inner catheter tube is closed at its distal end.

16. The zero-profile tip balloon catheter of claim 14, wherein the inner catheter tube is fixed to prevent movement within the outer catheter tube.

17. The zero-profile tip balloon catheter of claim 14, wherein the inner catheter tube is movable within the outer catheter tube to shorten or lengthen the distance between the proximal end region and the distal end region of the inflatable balloon structure.

18. The zero-profile tip balloon catheter of claim 10, wherein the middle region, proximal end region, and distal end region of the inflatable balloon structure comprise the same material composition.

19. A zero-profile balloon device comprising:
an inflatable balloon structure comprising a middle region, a proximal end region with a proximal opening, and a distal end region with a distal opening, wherein the distal end region is proximally invertible and passable through said proximal opening, and wherein the middle region and distal end region are sufficiently flexible to permit the distal end region to be inverted and passed through the proximal opening;
a proximal sealing component connectively coupled to said inflatable balloon structure at the proximal end region; and
a distal sealing component connectively coupled to said inflatable balloon structure at the distal end region such that the distal end region of the inflatable balloon structure distally extends beyond the distal end of the distal sealing component, wherein the distal end region is inverted where connectively coupled to said distal sealing component.

20. The zero-profile balloon device of claim 19, wherein the distal sealing component is one of a plug, a cap, or a tubular obstruction.

21. The zero-profile balloon device of claim 19, wherein the proximal sealing component is one of a band, a collar, or a pinch.

22. The zero-profile balloon device of claim 19, wherein the proximal sealing component comprises an inflation coupling for use in inflating or deflating the inflatable balloon structure.

23. The zero-profile balloon device of claim 19, wherein the proximal sealing component comprises at least one inflation vent for use in inflating or deflating the inflatable balloon structure.

24. The zero-profile balloon device of claim 19, further comprising a minimum spacing device internal to the inflatable balloon structure that maintains apart at a minimum distance the distal sealing component and the proximal sealing component.

25. The zero-profile balloon device of claim 19, further comprising a maximum spacing device internal to the inflatable balloon structure that maintains together at a maximum distance the distal sealing component and the proximal sealing component.

26. The zero-profile balloon device of claim 19, wherein the middle region, proximal end region, and distal end region of the inflatable balloon structure comprise the same material composition.

27. A method for inverting a distal end of an inflatable balloon structure, said inflatable balloon structure having a middle region, a first end region with a first opening, and a second end region with a second opening, said method comprising:
centrally inverting the second end region of the inflatable balloon structure and passing it through the first opening;
permanently fixing the inverted second end region to prevent un-inversion; and
returning the second end region back through the first opening.

28. The method of claim 27, wherein the first opening is larger than the second opening.
